(19) United States
(12) Patent Application Publication
(10) Pub. No.: US 2009/0281194 A1
(43) Pub. Date: Nov. 12, 2009

(54) Combinations for treating HIV-associated pain

(76) Inventors: Donald Johns, Woburn, MA (US); Thomas George Evans, Shanghai (CN)

Correspondence Address: FOLEY AND LARDNER LLP;SUITE 500, 3000 K STREET NW, WASHINGTON, DC, 20007, US

(21) Appl. No.: 12/395,980

(22) Filed: Mar. 2, 2009

(30) Foreign Application Priority Data
Feb. 28, 2008 (EP) 08152104.9

(51) Int. Cl. A61K 31/12; A61K 31/137; A61P 25/00

(52) U.S. Cl. 514/648; 514/682

(57) ABSTRACT

The present invention relates to novel combinations suitable for the treatment or amelioration of pain of various genesis or aetiology, which comprise, as active ingredients, at least one cannabinoid receptor binding compound, in particular a cannabinoid receptor binding naphthalene derivative, and at least one opioid, to their preparation, to their use as medicaments and to medicaments comprising them.

The present invention relates to combinations, which comprise at least two different organic compounds, to their preparation, to their use as medicaments and to medicaments comprising them.

More particularly, the present invention relates to novel combinations suitable for the treatment of pain of various genesis or aetiology, which comprise, as active ingredients, at least one cannabinoid receptor binding compound, in particular a cannabinoid receptor binding naphthalene derivative, and at least one opioid.

Cannabinoid receptor binding naphthalene derivatives are a class of compounds described, e.g., in WO-2002/42248, the contents of which publication are herewith incorporated hereinto by reference.

A preferred cannabinoid receptor binding naphthalene derivative is a compound of the formula

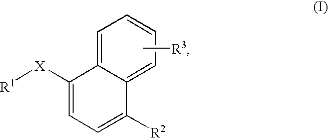

wherein
X is —S—, —S(═O)—, —S(═O)₂—, —S(═O)₂N(H)—, —P(═O)(OCH₃)—, —P(═O)(OH)—, —N(H)—, —N(CH₃)—, —N(H)C(═O)N(H)—, —C(═O)—, —C(═O)O—, —N(H)C(═O)—, —CH(OH)—, —CH═N—, —CH═CH—, —CH₂N(H)— or —C(═NH)—;
R¹ is aryl or heteroaryl;
R² is hydrogen, OR⁴ or N(R⁵)R⁶;
   R⁴ is C₁-C₈alkyl or C₂-C₈alkenyl;
   R⁵ and R⁶ independently are hydrogen, C₁-C₈alkyl or C(═O)C₁-C₈alkyl; and
R³ is hydrogen, cyano, heteroaryl, heterocycloalkyl, C(═O)R⁷, OR⁸ or N(R⁹)R¹⁰;
   R⁷ is OH, C₁-C₄alkoxy, NH₂, N(H)CH₂C(═O)OH or aryl;
   R⁸ is hydrogen, C₁-C₈alkyl, C(═O)C₁-C₄alkyl or C(═O)-aryl; and
   R⁹ and R¹⁰ independently are hydrogen, C₁-C₈alkyl or C₂-C₄alkenyl;
      with the proviso, that, when X is —C(═O)— and R² and R³ are hydrogen or R² is H and R³ is 4-methoxy, R¹ is neither 1-naphthyl nor 4-methoxy-1-naphtyl;
      in free form or in pharmaceutically acceptable salt form.

Aryl or heteroaryl is to be understood to include a five- or six-membered ring or a bicyclic system consisting of two six-membered rings or one five-membered and one six-membered ring, wherein one or more of the ring carbon atoms may be replaced, independently of one another, by a ring hetero atom selected from the group, consisting of oxygen, nitrogen and sulfur. Examples include C₆-C₁₀aryl, C₅-C₁₀heteroaryl, and C₆aryl condensed to a five- or six-membered aliphatic or heteroaliphatic ring, e.g. naphthyl, 1,2,3,4-tetrahydronaphthyl, phenyl, indolyl, quinolyl, isoquinolyl, 1,2,3,4-tetrahydroquinolyl, benzothiazolyl, imidazolyl, benzimidazolyl, benzoxadiazolyl, benzotriazolyl, indanyl, oxadiazolyl, pyrazolyl, triazolyl or tetrazolyl.

Examples for heterocycloalkyl include piperidyl, piperazinyl and morpholinyl.

The above defined compounds may bear substituents, e.g. one or more substituents, selected from OH; nitro; halogen; cyano; C(═O)OH; C(═O)NH₂; CI═O)N(H)N(H)C(═O)CH₃; C(NH₂)═NOH; C₁-C₄alkyl; S—C₁-C₄alkyl; C₁-C₈alkoxy; C₆-C₁₀aryl, such as phenyl; C₅-C₁₀-heteroaryl, such as oxadiazolyl; N-heterocycloalkyl, such as morpholinyl or piperidyl; C(═O)O—C₁-C₄alkyl; or N(R¹¹)R¹², wherein R¹¹ and R¹² independently are hydrogen, C₁-C₄alkyl, C(═O)N(H)O—C₁-C₄alkyl, C(═O)C₁-C₄alkyl or S(═O)₂—C₁-C₄alkyl; which substituents again may be substituted by a substituent, selected from OH; nitro; NH₂; C₁-C₄alkyl; C₁-C₄alkoxy; C₁-C₄alkoxy substituted by OH; C₃-C₆cycloalkyl; N—(C₁-C₄alkyl)₂; phenyl; or morpholinyl.

A cannabinoid receptor binding naphthalene derivative especially preferred according to the invention is naphthalen-1-yl-(4-pentyloxynaphthalen-1-yl)-methanone described in WO-2002/42248, i.e. the compound of the formula

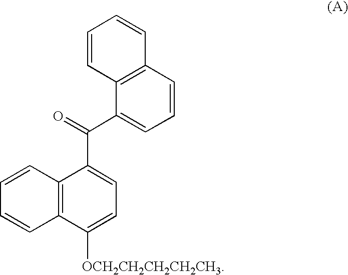

A cannabinoid receptor binding naphthalene derivative may be prepared as described in WO-2002/42248.

The term “opioid” as used herein refers to all substances, both natural and synthetic, with morphine-like actions. An opioid suitable for the present invention is especially selected from the group, consisting of alfentanil, allylprodine, alphaprodine, anileridine, benzylmorphine, bezitramide, buprenorphine, butorphanol, clonitazene, codeine, cyclorphan, desomorphine, dextromoramide, dezocine, diampromide, dihydrocodeine, dihydromorphine, eptazocine, ethylmorphine, fentanyl, hydrocodone, hydromorphone, hydroxypethidine, levophenacylmorphane, levorphanol, lofentanil, methadone, methyl morphine, morphine, necomorphine, normethadone, normorphine, opium, oxycodone, oxymorphone, pholcodine, profadol and sufentanil.

An opioid especially preferred according to the invention is methadone.

For example, alfentanil can be used, e.g., in the form as marketed, e.g. under the trademark Rapifen™; allylprodine can be used, e.g., in the form as marketed, e.g. under the trademark Alperidine™; anileridine can be used, e.g., in the form as marketed, e.g. under the trademark Leritine™; benzylmorphine can be used, e.g., in the form as marketed, e.g. under the trademark Peronine™ bezitramide can be used, e.g., in the form as marketed, e.g. under the trademark Burgodin™; buprenorphine can be used, e.g., in the form as marketed, e.g. under the trademark Buprenex™; butorphanol can be used, e.g., in the form as marketed, e.g. under the trademark Torate™; dextromoramide can be used, e.g., in the form as marketed, e.g. under the trademark Palfium™ dezocine can be used, e.g., in the form as marketed, e.g. under the trademark Dalgan™; dihydrocodeine can be used, e.g., in the form as marketed, e.g. under the trademark Novicodin™; dihydromorphine can be used, e.g., in the form as marketed, e.g. under the trademark Paramorphan™; eptazocine can be used, e.g., in the form as marketed, e.g. under the trademark Sedapain™; ethylmorphine can be used, e.g., in the form as marketed, e.g. under the trademark Dionin™; fentanyl can be used, e.g., in the form as marketed, e.g. under the trademark Fentanest™ eptanal; hydrocodone can be used, e.g., in the form as marketed, e.g. under the trademark Bekadid™ or Calmodid™; hydromorphone can be used, e.g., in the form as marketed, e.g. under the trademark Novolaudon™; hydroxypethidine can be used, e.g., in the form as marketed, e.g. under the trademark Bemidone™; levorphanol can be used, e.g., in the form as marketed, e.g. under the trademark Dromoran™; methadone can be used, e.g., in the form as marketed, e.g. under the trademark Dolophine™ or Methadose™; normethadone can be used, e.g., in the form as marketed, e.g. under the trademark Ticarda™; oxycodone can be used, e.g., in the form as marketed, e.g. under the trademark Dihydrone™ and oxymorphone can be used, e.g., in the form as marketed, e.g. under the trademark Numorphan™.

The structure of an active ingredient identified by a generic or trade name may be taken from the current edition of a standard compendium, e.g. “The Merck Index”, or from a database, e.g. Patents International (e.g. IMS World Publications). The corresponding contents thereof are herewith incorporated hereinto by reference. Any person skilled in the art is fully enabled to identify the active ingredients based on these references.

In particular, a cannabinoid (CB) receptor binding naphthalene derivative exhibits CB receptor binding activity at the human CB₁ receptor. CB receptor interaction may be demonstrated, e.g., by the ability to displace [³H]CP55940 from human CB receptors expressed in, e.g., pEAK cells, e.g. as demonstrated in accordance with the test method described in Example 1.

As disclosed in WO-02/42248, the CB receptor is a suitable target for the development of new medicaments to treat or ameliorate pain. Hence, a modulator, in particular an agonist, of the CB receptor activity can be used to treat or ameliorate pain.

The endogenous opioid system is a major inhibitory system in the central nervous system and plays a pivotal role in the modulation of pain. Activation of opioid receptors (p, kappa and 6) results in analgesia and anti-hyperalgesia in experimental models and in the clinic. Hence, an opioid can be used to treat or ameliorate pain. The use of opioids is affected by a number of known side-effects and disadvantages, such as a decrease in attention and concentration due to sedation, constipation and respiratory depression after taking the drug as well as the risk of drug abuse and drug addiction.

Analgesic activity may be confirmed in accordance with standard test methods, e.g. as described in Example 2.

Surprisingly, it has been found, that a combination, which comprises a cannabinoid receptor binding naphthalene derivative and an opioid, is advantageous in the treatment or amelioration of pain.

Hence, the invention relates to a combination, such as a combined preparation or pharmaceutical composition, which comprises at least one cannabinoid receptor binding compound, in particular a cannabinoid receptor binding naphthalene derivative, as the first active ingredient and at least one opioid as the second active ingredient, in which the active ingredients are present in each case in free form or in the form of a pharmaceutically acceptable salt, and optionally at least one pharmaceutically acceptable carrier, for simultaneous, separate or sequential use.

It will be understood, that a reference to an active ingredient is meant to also include its pharmaceutically acceptable salts. If an active ingredient has, e.g., at least one basic center, it can form an acid addition salt. An active ingredient having at least one acidic group can form a salt with a base.

An active ingredient, in free form or in pharmaceutically acceptable salt form, may be in the form of a hydrate and/or may include other solvents, for example solvents used for the crystallization of a compound in solid form.

The terms “pain” and “pain of various genesis or aetiology” as used herein include, but are not limited to, inflammatory pain, hyperalgesia and, in particular, chronic pain, and mean in particular pain consequential to trauma, e.g. associated with burns, sprains, fractures or the like, pain subsequent to surgical intervention, e.g. post-operative pain, chemotherapy-induced pain, as well as inflammatory pain of diverse genesis, e.g. bone and joint pain (e.g. osteoarthritis, rheumatoid arthritis or rheumatic disease), myofascial pain (e.g. muscular injury or fibromyalgia), lower back pain, chronic inflammatory pain, neuropathic, e.g. chronic neuropathic, pain (e.g. diabetic neuropathy, post-herpetic neuralgia or phantom limb pain), perioperative pain (e.g. associated with general surgery or gynecologic surgery), HIV associated pain [e.g. HIV associated neuropathic pain, HIV associated neuropathy, painful HIV associated neuropathy, HIV associated painful peripheral neuropathy, HIV associated distal sensory polyneuropathy (DSP) or antiretroviral toxic neuropathy (ATN)] or pain associated with, e.g., angina, menstruation or cancer.

Preferably, the terms “pain” and “pain of various genesis or aetiology” mean HIV associated pain, e.g. HIV associated neuropathic pain, HIV associated neuropathy, painful HIV associated neuropathy, HIV associated painful peripheral neuropathy, HIV associated distal sensory polyneuropathy (DSP) or antiretroviral toxic neuropathy (ATN).

Preferably, the terms “pain” and “pain of various genesis or aetiology” mean HIV associated pain, e.g. painful HIV associated neuropathy or HIV associated distal sensory polyneuropathy (DSP).

Preferably, the terms “pain” and “pain of various genesis or aetiology” mean painful HIV-associated neuropathy (including HIV-associated distal sensory polyneuropathy [DSP] and antiretroviral toxic neuropathy [ATN]).

The terms “treatment”, “treat” and “treating” as used herein include corresponding preventive activities.

The term “a combined preparation” as used herein defines especially a “kit of parts” in the sense, that the first and the second active ingredients as defined above can be dosed independently, either in separate form or by use of different fixed combinations with distinguished amounts of the active ingredients. The ratio of the amount of the active ingredient 1 to the amount of the active ingredient 2 to be administered in the combined preparation can be varied, e.g. in order to cope with the needs of a patient sub-population to be treated or the needs of a single patient, which needs can be different due to age, sex, body weight, etc. of a patient. The parts of the kit of parts can be administered simultaneously or chronologically staggered, e.g. at different time points and with equal or different time intervals for any part of the kit of parts. Preferably, the administration scheme is chosen in such a way, that the effect on the disease in the case of the combined use of the parts is larger than the effect, which would be obtained by use of only any one of the active ingredients. Preferably, there is at least one beneficial effect, e.g. an enhancing of the effect of the first and/or of the effect of the second active ingredient, in particular a synergism, e.g. a more than additive effect, an additional advantageous effect, fewer or weaker side effects or a combined therapeutical effect at a non-effective dosage of one or both of the first and the second active ingredients.

A combination, which comprises at least one cannabinoid receptor binding naphthalene derivative and at least one opioid, in which the active ingredients are present in each case in free form or in the form of a pharmaceutically acceptable salt, will be referred to hereinafter as a COMBINATION OF THE INVENTION.

Preferably, a COMBINATION OF THE INVENTION comprises a cannabinoid receptor binding naphthalene derivative of the formula I, especially of the formula A, and methadone.

Surprisingly, it was found, that the administration of a COMBINATION OF THE INVENTION results in a beneficial, e.g. a synergistic, therapeutic effect or in other surprising beneficial effects, e.g. in fewer or weaker side effects, compared to a monotherapy applying only one of the active ingredients used in the COMBINATION OF THE INVENTION.

In particular, a COMBINATION OF THE INVENTION, which comprises subeffective doses of a cannabinoid receptor binding naphthalene derivative and of an opioid, may achieve the same effect as effective doses of either compound alone.

In particular, by means of the administration of a COMBINATION OF THE INVENTION it is possible to alleviate the adverse side effect profile of opiods by allowing dose sparing through use of such COMBINATION OF THE INVENTION.

A further benefit is, that lower doses of the active ingredients of the COMBINATION OF THE INVENTION can be used, compared to a monotherapy applying only one of the active ingredients used in the COMBINATION OF THE INVENTION. For example, the dosages used may not only be smaller, but may also be applied less frequently. Preferably, the incidence of side effects may be diminished. This is in accordance with the desire and requirements of the patient to be treated.

The pharmacological activity of a COMBINATION OF THE INVENTION for the treatment or amelioration of pain may, for example, be shown in test models known as such, e.g. in those described in the Examples, or be demonstrated in clinical studies. Such clinical studies are preferably randomized, double-blind, clinical studies in patients with chronic pain, e.g. post-herpetic neuralgia, diabetic neuropathy or cancer pain. Such studies may show, in particular, the synergism of the active ingredients of the COMBINATION OF THE INVENTION. The beneficial effects on pain can be determined directly through the results of these studies or via changes in the study design, which are known as such to a person skilled in the art. These studies are, in particular, suitable to compare the effects of a monotherapy using the active ingredients alone with the effects of a COMBINATION OF THE INVENTION.

The invention also relates to a pharmaceutical composition comprising a COMBINATION OF THE INVENTION as active ingredients and at least one pharmaceutically acceptable carrier. In this composition, the first and the second active ingredients can be administered together, one after the other or separately, in one combined unit dosage form or in two separate unit dosage forms. The unit dosage form may also be a fixed combination.

A pharmaceutical composition according to the invention is, preferably, suitable for enteral, such as oral or rectal, or parenteral administration to a mammal, including a human, and comprises a therapeutically effective amount of the active ingredients and one or more suitable pharmaceutically acceptable carriers. Preferred are compositions for oral, intravenous or nasal administration. A composition for enteral or parenteral administration is, for example, a unit dosage form, such as a sugar-coated tablet, a tablet, a capsule, a suppository or an ampoule. The unit content of active ingredients in an individual dose need not in itself constitute an effective amount, since such an amount can be reached by the administration of a plurality of dosage units. A composition according to the invention may contain, e.g., from about 10% to about 100%, preferably from about 20% to about 60%, of the active ingredients.

If not indicated otherwise, a pharmaceutical composition according to the invention is prepared in a manner known per se, e.g. by means of conventional mixing, granulating, sugarcoating, dissolving or lyophilizing processes. In preparing a composition for an oral dosage form, any of the usual pharmaceutical media may be employed, for example water, glycols, oils, alcohols, carriers, such as starches, sugars, or microcrystalline cellulose, diluents, granulating agents, lubricants, binders, disintegrating agents and the like. Because of their ease of administration, tablets and capsules represent the most advantageous oral dosage unit forms, in which case solid pharmaceutical carriers are obviously employed.

Furthermore, the invention relates to the use of a COMBINATION OF THE INVENTION for the preparation of a medicament for the treatment or amelioration of pain, especially chronic pain.

Furthermore, the invention relates to a method of treating or ameliorating pain, especially chronic pain, in a warm-blooded animal in need thereof, which comprises administering to the animal a therapeutically effective amount of a COMBINATION OF THE INVENTION. In particular, a therapeutically effective amount of each of the active ingredients of the COMBINATION OF THE INVENTION may be administered simultaneously or sequentially in any order, and the components may be administered separately or as a fixed combination. For example, the method of treating or ameliorating may comprise (i) the administration of the first active ingredient and (ii) the administration of the second active ingredient, simultaneously or sequentially in any order, in jointly therapeutically effective amounts, preferably in synergistically effective amounts. The individual active ingredients of the COMBINATION OF THE INVENTION can be administered separately at different times during the course of the therapy or concurrently in divided or single combination forms. The instant invention is to be understood as embracing all such regimes of simultaneous or alternating administration. Furthermore, the term “administering” also encompasses the use of a prodrug of an active ingredient, that is converted in vivo into the active ingredient.

The effective dosage of each of the active ingredients employed in the COMBINATION OF THE INVENTION may vary, depending on the particular active ingredient or pharmaceutical composition employed, the mode of administration, the severity of the condition to be treated, the age, sex, body weight, etc. of the patient, and the like. Thus, the dosage regimen for the COMBINATION OF THE INVENTION is selected in accordance with a variety of factors, including the renal and hepatic function of the patient. A physician, clinician or veterinarian of ordinary skill can readily determine and prescribe the appropriate dosage regimen. The dose of an opioid generally will be between about 75 ng and about 750 mg.

When an active ingredient employed in the COMBINATION OF THE INVENTION is applied in the form as marketed as monotherapy in pain, its dosage and mode of administration can take place in accordance with the information provided in the packet leaflet of the marketed product, if not mentioned otherwise herein.

Furthermore, the invention relates to a COMBINATION OF THE INVENTION for the use as a medicament.

Furthermore, the invention relates to a COMBINATION OF THE INVENTION for the treatment or amelioration of pain.

Furthermore, the invention relates to a COMBINATION OF THE INVENTION for the treatment or amelioration of HIV associated pain, e.g. painful HIV associated neuropathy or HIV associated distal sensory polyneuropathy (DSP).

Furthermore, the invention relates to a commercial package comprising a COMBINATION OF THE INVENTION as active ingredients and written instructions for the simultaneous, separate or sequential use thereof in the treatment or amelioration of pain.

Furthermore, the invention relates to a cannabinoid receptor binding naphthalene derivative of the formula I, especially of the formula A, for the treatment or amelioration of HIV associated pain, e.g. painful HIV associated neuropathy or HIV associated distal sensory polyneuropathy (DSP), and to a method of treating or ameliorating HIV associated pain, e.g. painful HIV associated neuropathy or HIV associated distal sensory polyneuropathy (DSP), comprising the administration of an effective amount of a derivative of the formula I, especially of the formula A.

The following Examples serve to illustrate the invention without limiting its scope.

### EXAMPLE 1

### CB1 Receptor Binding Assay

The assay mixture comprises 75 μl of membrane suspension [membranes from pEAK cells transfected with human CB1 receptors from Receptor Biology, Beltsville, Md.; 133 μg/ml in assay buffer (50 mM Tris-HCl, 2.5 mM EDTA, 5 mM MgCl₂ 5 mg/ml BSA, pH7.4), approximately 10 μg/well)], 25 μl of WGA-YS beads [Yftrium silicate beads coated with wheat germ agglutinin, Amersham (40 mg/ml, 1 mg/well)], 50 μl of test compound in 4% DMSO and 50 μl of radioligand {[³H]CP55940 (180 Ci/mmol), New England Nuclear; final concentration 0.125 nM, in assay buffer}. All components are mixed, shaken at room temperature for 2 hours and then counted on a Topcount. Non-saturable binding is measured in the presence of 10 μM (R)-(+)-[2,3-dihydro-5-methyl-3-[(4-morpholinyl)methyl]pyrrolo[1,2,3-de]-1,4-benzoxazin-6-yl]-(1-naphthyl)-methanone (Tocris). Kᵢ values are in the range of from 1 nM to 100 μM, preferentially from 10 nM to 2 μM. The IC₅₀ values are calculated in ORIGIN using a logistic fit. Kᵢ values are calculated from the IC₅₀ values using the Cheng-Prussoff equation (K=IC₅₀/(1+([L]/K_{d})), wherein [L] is the ligand concentration.

### EXAMPLE 2

### Neuropathic Pain Model

Hyperalgesia is examined in the model of neuropathic pain induced by partial ligation of the sciatic nerve as described by Seltzer et al. (1990). Briefly, Wistar rats (120 to 140 g) are anaesthetised, the left sciatic nerve is exposed at mid-thigh level through a small incision, and ⅓ to ½ of the nerve thickness is tightly ligated within a 7.0 silk suture. The wound is closed with a single muscle suture and skin clips and dusted with aureomycin antibiotic powder. The animals are allowed to recover and are used 12 to 15 days following surgery. Mechanical hyperalgesia is assessed by measuring paw withdrawal thresholds to an increasing pressure stimulus placed onto the dorsal surface of the paw using an analgesymeter (UgoBasile, Milan) with a cut-off of 250 g. Withdrawal thresholds are measured on both the ipsilateral (ligated) and contralateral (unligated) paw prior to (predose) and then up to 6 h following drug or vehicle administration. Data are expressed as withdrawal threshold (g) and percentage reversal of hyperalgesia calculated according to the following formula:

$$\% reversal = \frac{\begin{matrix}ipsilateral threshold postdose - \\ ipsilateral threshold predose\end{matrix}}{\begin{matrix}contralateral threshold predose - \\ ipsilateral threshold predose\end{matrix}} \times 100$$

Potency is expressed as D₅₀ value, i.e. the dose of drug necessary to produce a 50% reversal of hyperalgesia. Dfo values are in the range of from 0.1 to 100 mg/kg.

1. A combination, comprising:
a least one cannabinoid receptor binding compound as the first active ingredient and
at least one opioid as the second active ingredient in which the active ingredients are present in each case in free form or in the form of a pharmaceutically acceptable salt.

2. The combination according to claim 1, in which the cannabinoid receptor bending compound is a cannabinoid receptor binding naphthalene derivative of the formula 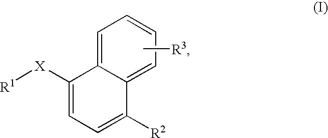
wherein
X is —S—, —S(═O)—, —S(═O)₂—, S(═O)₂N(H)—: —P(═O)(OCH₃)—, —P(═O)(OH)—, —N(H)—, —N(CH₃)—, —N(H)C(═O)N(H)—, —C(═O)O—, —N(H)C(═O)—, —CH(OH)—, —CH═N—, —CH═CH—, —CH₂N(H)— or —C(═NH)—;
R¹ is aryl or heteroaryl;
R² is hydrogen, OR⁴ or N(R⁵)R⁶;
R⁴ is C₁-C₈alkyl or C₂-C₈alkenyl;
R⁵ and R⁶ independently are hydrogen, C₁-C₆alkyl or C(═O)C₁-C₆alkyl; and
R³ is hydrogen, cyano, heteroaryl, heterocycloalkyl, C(═O)R⁷, OR⁸ or N(R⁹)R¹⁰;
R⁷ is OH, C₁-C₄alkoxy, NH₂, N(H)CH₂C(═O)OH or aryl; —
R⁸ is hydrogen, C₁-C₈alkyl, C(═O)C₁-C₄-alkyl or C(═O)-aryl; and
R⁹ and R¹⁰ independently are hydrogen, C₁-C₈alkyl or C₂-C₄alkenyl;
with the proviso, that, when X is —C(═O)— and R² and R³ are hydrogen or R² is H and R³ is 4-methoxy, R¹ is neither 1-naphthyl nor 4-methoxy-1-naphtyl;
in free form or In pharmaceutically acceptable salt form.

3. The combination according to claim 2, in which the cannabinoid receptor binding naphthalene derivative is a compound of the formula

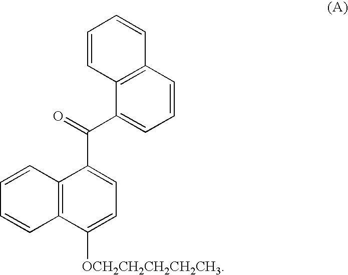

4. The combination according to claim 1, in which the opioid is selected from the group consisting of afentanil allylprodine, alphaprodine, anileridine benzylmorphine, ezitramide, buprenorphine, butorphanol, clonitazene, codeine, cyclorphan, desomorphine, dextromooramide, dezocine, diamprornide, dihydrocodeine, dihydromorphine, eptazocine, ethylnmorphine, fentanyl, hydrocodone, hydromorphone, hydroxpethidne, levophenacylmorphane, levorphanol, lofentanil, methadone methymorphine, morphine, necomorphine, normethadone, normorphine, opium, oxycodone, oxymorphone, pholcodine, profadol and sufentanil.

5. The combination according to claim 3, in which the opioid is methadone.

6. A pharmaceutical composition, comprising:
the combination as defined in claim 1 as active ingredients and
at least one pharmaceutically acceptable carrier.

7-9. (canceled)

10. A commercial package, comprising:
the combination as defined in claim 1 as active ingredients and
written instructions for the simultaneous, separate or sequential use thereof in the treatmnent or amelioration of pain.

11. (canceled)

12. A method of treating or ameliorating pain in a warm-blooded animal in need thereof; comprising:
administering to the animal a therapeutically effective amount of the combination as defined in claim 1.

13. (canceled)

14. A method of treating or ameliorating pain in a warm-blooded animal in need thereof, comprising:
administering to the animal a therapeutically effective amount of the compound of the formula A as defined in claim 3.
